(19) United States
(12) Patent Application Publication
(10) Pub. No.: US 2023/0091608 A1
(43) Pub. Date: Mar. 23, 2023

(54) COMPOUND AND DIAGNOSTIC SYSTEM COMPRISING SAID COMPOUND FOR THE GUSTATORY DETECTION OF INFLAMMATIONS IN THE ORAL CAVITY

(71) Applicant: Julius-Maximilians-Universitaet Wuerzburg, Wuerzburg (DE)

(72) Inventors: Lorenz MEINEL, Wuerzburg (DE); Jennifer RITZER, Wuerzburg (DE); Tobias MIESLER, Wuerzburg (DE)

(73) Assignee: Julius-Maximilians-Universitaet Wuerzburg, Wuerzburg (DE)

(21) Appl. No.: 17/440,319

(22) PCT Filed: Mar. 13, 2020
(86) PCT No.: PCT/EP2020/056873

§ 371 Date: Sep. 17, 2021

(30) Foreign Application Priority Data
Mar. 19, 2019 (EP) 19163922.8

(51) Int. Cl. C07C 237/04; A61K 49/00; C07K 7/06

(52) U.S. Cl. CPC C07C 237/04; A61K 49/0004; C07K 7/06

(57) ABSTRACT

The present invention relates to compounds comprising denatonium linked to the C-terminus of a protease-sensitive peptide; or a salt thereof. The compounds are useful in the diagnosis of inflammatory conditions of the oral cavity.

### PRIORITY

This application corresponds to the U.S. National phase of International Application No. PCT/EP2020/056873 filed Mar. 13, 2020, which, in turn, claims priority to European Patent Application No. 19163922.8 filed Mar. 19, 2019, the contents of which are incorporated by reference herein in their entirety.

### SEQUENCE LISTING

The instant application contains a Sequence Listing that has been submitted in ASCII format via EFS-Web and is hereby incorporated by reference in its entirety. Said ASCII copy, created on Sep. 17, 2021, is named LNK_231 US_SEQ_LIST.txt and is 3,605 bytes in size.

### BACKGROUND OF THE INVENTION

Inflammations in the oral cavity often occur in the form of swelling and redness, and can be extremely painful. Sometimes it comes to bleeding of the inflamed area. However, especially at an early stage, these inflammations often remain unrecognized by the patients or the severe consequences, if not treated, are underestimated.

If the gums (gingiva) are affected by an inflammation such as periodontitis and peri-implantitis, for example, this can lead to a loosening of the teeth followed by tooth loss in the longer term. Periodontal disease is a bacterial inflammation, which manifests itself in a largely irreversible destruction of the periodontium. It is associated with a loss of holding due to degradation of the periodontium. Peri-implantitis is a destructive inflammatory process affecting the soft and hard tissues surrounding dental implants. The soft tissues become inflamed whereas the alveolar bone (hard tissue), which surrounds the implant for the purposes of retention, is lost overtime. Pain when swallowing, increased salivation, bad breath and an unpleasant taste in the mouth are possible accompanying symptoms. Sometimes associated with fever.

Medical advice is often consulted only when the symptoms become manifest and it is too late for timely treatment. Sometimes, these inflammations are only diagnosed by chance, for example when a patient visits a dentist due to pain because of another condition such as tooth decay. Accordingly, there is always the risk that inflammations in the oral cavity are missed in patients who for some reason do not go to see a dentist on a regular basis.

Nevertheless, early detection and treatment of these diseases is particularly important as they are suspected of affecting the course of other diseases such as diabetes or chronic ischemic cardiovascular disease for the patient adversely or even cause other complications of the general health themselves.

It is therefore desired to find a reliable, specific, uncomplicated and rapid way of diagnosing these diseases at the earliest possible stage, preferably by the patient himself without having to consult a dentist. Suitable solutions include easy to use products for daily use. New rapid tests based on bio-responsive systems reduce costs, time and complexity and can therefore be used for a first classification.

Bioresponsive systems react to changes in the environment with the release of substances. This principle is used on the one hand therapeutically, but can also be used in the diagnosis of diseases. In case of a disease otherwise stable parameters of the organism change, which is noticed by these systems and as a result of which an easily detectable indicator substance is released.

In this context, US. Pat. App. Pub. No. US 2011/0081673 discloses a diagnostic chewing gum for diabetes screening. The patent application teaches a diagnostic chewing gum for screening a medical condition such as diabetes due to a detectable change in color based on whether the patient is healthy or not. Thereby, the intensity of the color change may indicate a degree of seriousness of a medical condition or a degree of risk for a medical condition.

Another disclosure PCT Pat. Pub. No. WO 2013/131993 relates to a device for the diagnosis of inflammatory tissues in dental applications. Specifically, said disclosure teaches a diagnostic chewing gum for identifying the presence of inflammatory tissues in the mouth, in particular in or adjacent to the mandible, the maxilla, an implant or the teeth of a user. Also, U.S. Pat. No. 9,526,803 provides an approach for the direct detection of pathogens (viruses, bacteria, fungi and combinations thereof) in the mouth and adjacent tissues. This is done by means of identifying the presence of the pathogens via the mouth with the chewing gum.

In both PCT Pat. Pub. No. WO 2013/131993 and U.S. Pat. No. 9,526,803 a poly(methylmethacrylate) (PMMA) substrate and/or anchor for attachment of the flavoring or colorant substance via a linker is required.

A similar sensor for the diagnosis of periodontal disease and peri-implantitis is explained by Ritzer, J et al. (Nat. Commun. Diagnosing peri-implant disease using the tongue as a 24/7 detector. volume 8, Article number: 264 (2017)). Therein, it is reported on sensory chewing gums as a bioresponsive system for the detection of periodontal disease and peri-implantitis. The bioresponsive system contains peptide sensors consisting of a protease-sensitive peptide linker ((PSL), also referred to as protease cleavage linker (PCL)) between a bitter flavoring substance such as a denatonium compound and a PMMA microparticle.

Denatonium is the bitterest compound currently known. Due to its extremely bitter taste, it is used in cosmetics and household products as an additive to prevent inadvertent ingestion. A medical use of the bitter substance is the addition in nail polish against nail biting.

Scheme A is a graphical representation of the diagnostic system according to Ritzer, J et al. Therein, the flavoring compound (denatonium-COOH) is connected to the N-terminus of the PSL via the carboxyl group of the denatonium-COOH. Subsequently, the product is connected to the PMMA particle via the PSL's azide group.

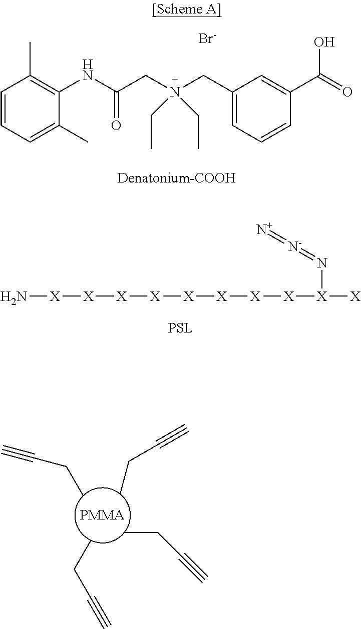

In a healthy patient, the large (uncleaved) intact bioresponsive sensor is water insoluble and tasteless. If a disease is present, the sensor is specifically cleaved by disease-induced matrix metalloproteinases (MMP) in the patient saliva. This cleavage results in low molecular weight, water soluble and bitter substances. It is for this bitterness that the patient is alarmed by recognizing a strong taste. Comprised in a chewing gum, the system may be used for the diagnosis of e.g. periodontal disease and peri-implantitis.

When the bioresponsive system is incubated with MMP, it is cleaved quickly and specifically at a peptide function of the PSL into two segments with four remaining amino acids at the denatonium-COOH, as it is exemplified in Scheme B.

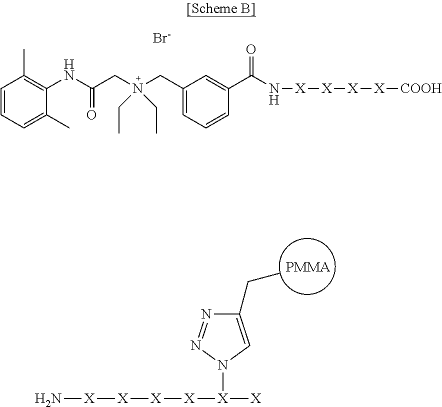

Subsequently also the other amino acids are cleaved off. Finally, only the denatonium-COOH remains, which can be detected by the tongue in low concentrations due to its bitterness.

So far, the denatonium-peptide constructs were coupled to PMMA particles in order to mask the bitter taste. This is because the known denatonium-peptide constructs were gustatory detectable, unless linked to a PMMA particle. The PMMA particle prevented the gustatory detection due to its size and insolubility in water.

The inventors of the present invention have surprisingly found that the gustatory detection of the denatonium compound is already effectively blocked by a four-amino acid peptide, if the denatonium compound is linked to the C-terminus of the peptide via an amine function. Therefore, compared to the prior art, no PMMA particles are required anymore for the bioresponsive sensors. Accordingly, the inventive bioresponsive sensor can be prepared faster, cheaper and more easily. It was further found by the inventors that it is difficult to provide stable amino-functionalized denatonium derivatives that can be coupled to peptides via their C-terminal carboxyl group.

Modified denatonium compounds are provided that can be attached to the C-terminus of a protease-sensitive peptide so that a bioresponsive sensor can be prepared that enables the patient to diagnose inflammations in the oral cavity such as periodontal disease and/or perk implantitis by himself.

### SUMMARY OF THE PRESENT INVENTION

In summary, the present invention is directed to the following items [1] to [41]:
[1] A compound having the structure Den-R¹—NH—R², or a salt thereof, wherein
   Den is denatonium,
   R¹ is an optionally substituted C₁₋₃ alkylene group
   R² is a protease-sensitive peptide,
   wherein the C-terminus of the protease-sensitive peptide forms an amide bond with the group —NH—.
[2] The compound or salt of item [1] having the formula [I]:

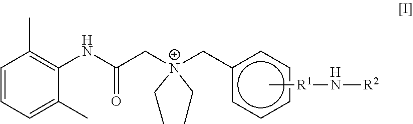

wherein
R¹ and R² are as defined in item [1].
[3] The compound or salt of item [1] or [2], having the formula [Ia]:

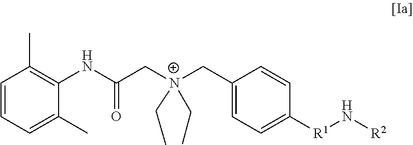

wherein
R¹ and R² are as defined in item [1].
[4] The compound or salt of any one of items [1] to [3], wherein R¹ is —CH₂—.
[5] The compound or salt of any one of items [1] to [4], wherein the protease-sensitive peptide comprises or consists of at least 4 amino acids, preferably 4 to 15 amino acids, more preferably 5 to 12, even more preferably 6 to 10, yet even more preferably 7 to 9, most preferably 8 amino acids.
[6] The compound or salt of any one of items [1] to [5], wherein the C-terminal amino acid of the protease-sensitive peptide is selected from the group consisting of alanine, arginine, glutamine, leucine, methionine and phenylalanine.
[7] The compound or salt of any one of items [1] to [5], wherein the C-terminal amino acid of the protease-sensitive peptide is not alanine or valine.
[8] The compound or salt of any one of items [1] to [5], wherein the C-terminal amino acid of the protease-sensitive peptide is glutamine.
[9] The compound or salt of any one of items [1] to [5], wherein the C-terminal amino acid of the protease-sensitive peptide is alanine.
[10] The compound or salt of any one of items [1] to [5], wherein the C-terminal amino acid of the protease-sensitive peptide is arginine.
[11] The compound or salt of any one of items [1] to [5], wherein the C-terminal amino acid of the protease-sensitive peptide is tyrosine.
[12] The compound or salt of any one of items [1] to [5], wherein the C-terminal amino acid of the protease-sensitive peptide is glutamic acid.
[13] The compound or salt of any one of items [1] to [5], wherein the C-terminal amino acid of the protease-sensitive peptide is leucine.
[14] The compound or salt of any one of items [1] to [5], wherein the C-terminal amino acid of the protease-sensitive peptide is serine.
[15] The compound or salt of any one of items [1] to [5], wherein the C-terminal amino acid of the protease-sensitive peptide is methionine.
[16] The compound or salt of any one of items [1] to [5], wherein the C-terminal amino acid of the peptide is phenylalanine.
[17] The compound or salt of any one of the preceding items, wherein the protease-sensitive peptide is not QPVV, DAPV or GPQGIAGA.
[18] The compound or salt of any one of the preceding items, wherein the protease-sensitive peptide is susceptible to cleavage by a matrix metalloproteinase.
[19] The compound or salt of any one of the preceding items, wherein the protease-sensitive peptide is susceptible to cleavage by a matrix metalloproteinase-8 or activated matrix metalloproteinase-8.
[20] The compound or salt of any one of the preceding items, wherein the protease-sensitive peptide is susceptible to cleavage by a pathogen-specific protease.
[21] The compound or salt of item [20], wherein the pathogen is a bacterial pathogen.
[21] The compound or salt of item [20], wherein the pathogen is a viral pathogen.
[22] The compound or salt of any one of the preceding items, wherein the protease is an endopeptidase
[23] The compound or salt of any one of the preceding items, wherein the compound is not susceptible to cleavage by an aminopeptidase.
[24] The compound or salt of any one of the preceding items, wherein the compound is not susceptible to cleavage by an exopeptidase.
[25] The compound or salt of any one of the preceding items, wherein the protease-sensitive peptide has a length of 5 to 20 amino acids.
[26] The compound or salt of any one of the preceding items, wherein the protease-sensitive peptide has a length of 6 to 18 amino acids.
[27] The compound or salt of any one of the preceding items, wherein the protease-sensitive peptide has a length of 7 to 16 amino acids.
[28] The compound or salt of any one of the preceding items, wherein the protease-sensitive peptide has a length of 8 to 14 amino acids.
[29] The compound or salt of any one of the preceding items, wherein the protease-sensitive peptide has a length of 9 to 12 amino acids.
[30] The compound or salt of any of the preceding items, having the formula [BRS-1]:

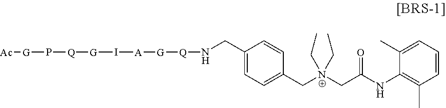

[31] The salt of any one of the preceding items, further comprising a counter ion selected from the group consisting of
   TFA⁻, F⁻, Cl⁻, Br⁻,

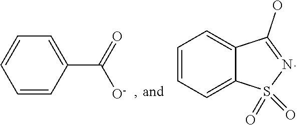 [SMILES: O=C([O-])c1ccccc1 ; O=S1(=O)N=C(O)c2ccccc21]

[32] A bioresponsive sensor comprising the compound or salt of any one of the preceding items.
[33] The compound or salt according to any one of items [1] to [31], or the bioresponsive sensor according to item [32] for use in a method of detecting an inflammation in the oral cavity of a human patient.
[34] The compound or salt for use according to item [33], or the bioresponsive sensor for use according to item [33], wherein the inflammation is detected by gustatory perception by the patient.
[35] The compound or salt for use according to item [33] or [34], or the bioresponsive sensor for use according to item [33] or [34], wherein the inflammation is detected upon cleavage of the compound, thereby releasing the denatonium.
[36] The compound or salt for use according to any one of items [33] to [35], or the bioresponsive sensor for use according to item [33] to [35], wherein, upon cleavage of the compound, a compound having the formula [II]:

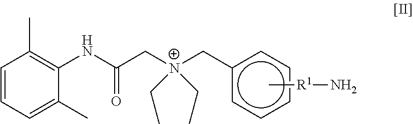

is released,
wherein R¹ is an optionally substituted C₁₋₃ alkylene group.
[37] The compound or salt for use according to item [36], or the bioresponsive sensor for use according to item [36], wherein the compound of formula [II] is a compound having the formula [IIa-1]:

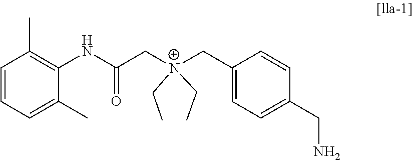 [SMILES: CC[N+](CC)(CC(=O)Nc1c(C)cccc1C)Cc1ccc(CN)cc1]

[38] A method for the preparation of the bioresponsive sensor, comprising the step of linking an amino-modified denatonium compound to the C-terminus of a protease-sensitive peptide, wherein the amino-modified denatonium compound is preferably a compound of formula [II] or formula [IIa-1].
[39] The method of item [38], further comprising, prior to the step of linking:
   preparing a protease-sensitive peptide by solid phase peptide synthesis, and/or
   protecting the N-terminus of the protease-sensitive peptide with a protecting group, preferably acetic anhydride.
[40] A diagnostic chewing gum or a diagnostic confectionary, comprising the compound or salt of any one of items [1] to [31].
[41] A compound of formula [II]:

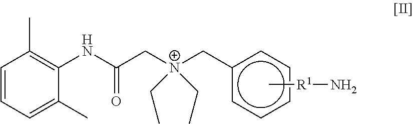

wherein R¹ is an optionally substituted C₁₋₃ alkylene group;
with the proviso that the compound of formula [IIa-1]

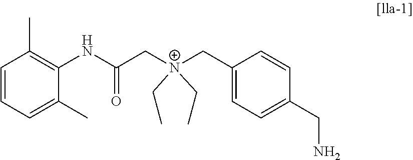 [SMILES: CC[N+](CC)(CC(=O)Nc1c(C)cccc1C)Cc1ccc(CN)cc1]

is excluded.

### BRIEF DESCRIPTION OF THE FIGURES

FIG. 1 shows as graphical representation of the cleavage rate of [BRS-1] and [CC1] accompanied by the standard deviations.

FIG. 2 is a graphical representation of the cleavage rate of [IIa-1]+AA of 16 of the 20 proteinogenic amino acids.

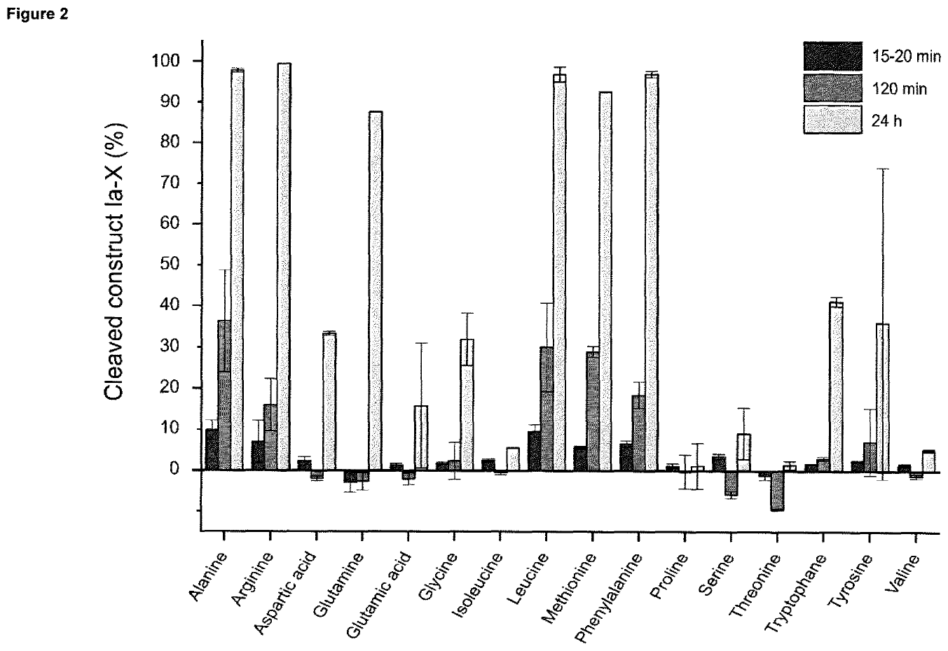

### DETAILED DESCRIPTION OF THE PRESENT INVENTION

In a first aspect, the present invention relates to a compound comprising denatonium linked to the C-terminus of a protease-sensitive peptide; or a salt thereof. Preferably, the compound is a compound according to formula [I]:

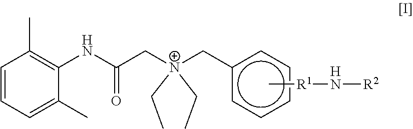

wherein

R¹ is an optionally substituted C₁₋₃ alkylene group, and

R² is the protease-sensitive peptide; or a salt thereof. Preferably R¹ is an unsubstituted C₁₋₃ alkylene group. More preferably R¹ is methylene, ethylene or propylene. Most preferably, R¹ is methylene.

Preferred embodiments of the compound according to formula [I] are selected from the following group consisting of [Ia] to [Ic], wherein [Ia] is particularly preferred:

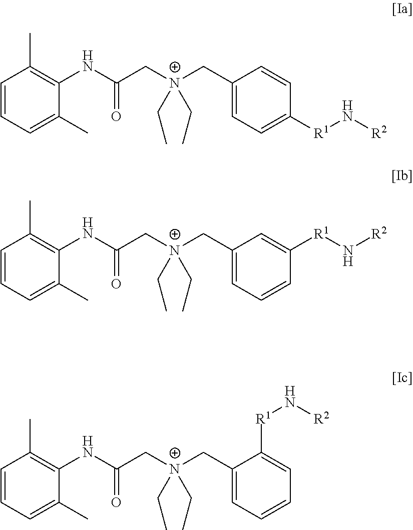

wherein R¹ and R² are as defined above.

In one embodiment, the compound according to formula [Ia] is selected from the group consisting of [Ia-1] to [Ia-3], wherein formula [Ia-1] is preferred:

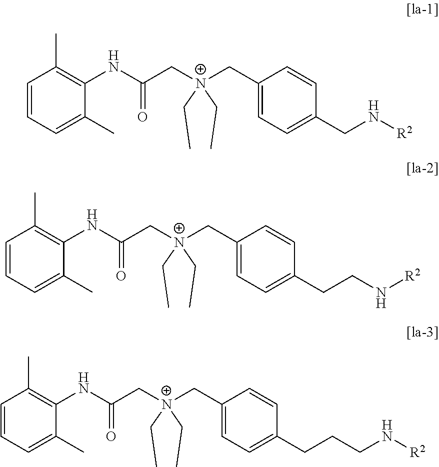

wherein R² is the protease-sensitive peptide.

In another embodiment, the compound according to formula [Ib] is selected from the group consisting of [Ib-1] to [Ib-3]:

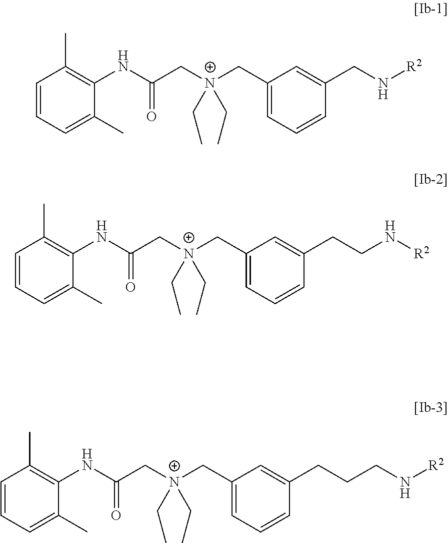

wherein R² is the protease-sensitive peptide.

In another embodiment, the bioresponsive sensor according to formula [Ic] is selected from the group consisting of [Ic-1] to [Ic-3]:

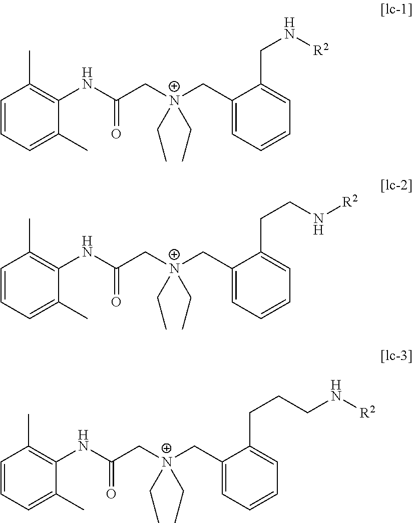

wherein R² is the protease-sensitive peptide.

The amino acid sequence of the protease-sensitive peptide is not particularly limited as long as it is susceptible to cleavage by an endopeptidease. Preferably the protease-sensitive peptide has a length of at least 4 amino acids, preferably it has a length of 4 to 15, more preferably 5 to 12, even more preferably 6 to 10, yet even more preferably 7 to 9, most preferably 8 amino acids.

The amino acids of the protease-sensitive peptide are independently selected from the group consisting of alanine (A), arginine (R), asparagine (N), aspartic acid (D), cysteine (C), glutamine (Q), glutamic acid (E), glycine (G), histidine (H), isoleucine (I), leucine (L), lysine (K), methionine (M), phenylalanine (F), proline (P), serine (S), threonine (T), tryptophan (W), tyrosine (Y) and valine (V).

In one embodiment, the amino acids are independently selected from the group consisting of alanine (A), arginine (R), aspartic acid (D), glutamine (Q), glutamic acid (E), glycine (G), isoleucine (I), leucine (L), methionine (M), phenylalanine (F), proline (P), serine (S), threonine (T), tryptophan (W), tyrosine (Y) and valine (V).

In another embodiment, the amino acids are independently selected from the group consisting of alanine (A), glutamine (Q), glycine (G), isoleucine (I), proline (P).

To obtain maximum gustatory perception, the amino acid directly attached to the nitrogen atom of the amino-modified denatonium, i.e. the C-terminal amino acid of the protease-sensitive peptide, is preferably selected from the group consisting of alanine (A), arginine (R), aspartic acid (D), glutamine (Q), glutamic acid (E), glycine (G), isoleucine (I), leucine (L), methionine (M), phenylalanine (F), proline (P), serine (S), threonine (T), tryptophan (W), tyrosine (Y) and valine (V).

More preferably, the C-terminal amino acid of the protease-sensitive peptide is selected from the group consisting of alanine (A), arginine (R), aspartic acid (D), glutamine (Q), glutamic acid (E), glycine (G), isoleucine (I), leucine (L), methionine (M), phenylalanine (F), serine (S), tryptophan (W) and tyrosine (Y).

More preferably, the C-terminal amino acid of the protease-sensitive peptide is selected from the group consisting of alanine (A), arginine (R), glutamine (Q), leucine (L), methionine (M) and phenylalanine (F).

In another preferred embodiment, the C-terminal amino acid of the protease-sensitive peptide is selected from the group consisting of alanine (A), arginine (R), glutamic acid (E), leucine (L), methionine (M) and phenylalanine (F).

In one embodiment, the protease is a pathogen-specific protease. The pathogen may be selected from the group consisting of virus, bacterium, protozoa, prion, fungus and combinations thereof. According to a preferred embodiment of the present invention, the protease or proteolytic enzyme is released or, in case of a virus or a prion, upregulated, by pathogens, preferably by bacteria, viruses, protozoa or fungi, more preferably the following class, order, genera, family of species of herpes, varicella, parvovirus, papillomavirus, polyomavirus, adenovirus, hepadnavirus, variolavirus, picornavirus, aso- and caliciavirus, human cytomegalovirus, hepatitis-A-virus, hepatitis-C-virus, hepatitis-E-virus, togavirus, flavivirus, coronavirus, retrovirus, HIV, reovirus, orthomyxovirus, bunyavirusarenavirus, human rhinovirus, dengue virus, varicella-zoster virus, paramyxovirus, rubulavoris, morbillivirus, west nile virus, yellow fever virus, pneimovirus, non classified paramyxovirus, rhabdovirus, folovirus, viroids and prions, staphylococcus, streptococcus and enterococcus, bacillus, listeria, erysipelothrix, garderella, corynebacterium, actinomyces, mycobacterium, nocardia, neisseria, acinetobacter and moraxella, enterbacteriacea including salmoneslla shigella, yersinia, E. coli and vibrio, aeromonas, plesiomonas, haemophilus, pasteurella, campyhlobacter, heliobacter, spirillum, pseudomonas, stenotropomonas, burkholderia, legionella, brucella, bordetella francisella, bacteriodaceae ioncluding trepponema, borrelia peptospira rickettsia, coxiella, orientia, ehrlichia, baronella afipia, chlamydia, mycoplasma and histoplasma, coccidioides, blasomyces, paracoccidioides, candida, aspergillus, Cryptococcus, mucor, absidia, rhizopus, phaeohyphomycetes, hyalohyphomycetes, penicillium, pneumocystis, tyrpanoma, leishmania, giradia, trichomonas, entamoeba, naegleria, toxoplasma isspora, cyclospora, sarcocystis, cryptosporidium, plasmodium, babesia, microsporida, and balantidium.

The protease may be selected from the group consisting of: KSHV-, HSV-, HAV-; HCV-, HIV-, human cytomegalovirus-, Yellow fever, CMV-, HRV14-, HRV2a-, Malaria aspartyl-, Sars protease, proteases of the S1, S2, S6, S8, S9, S33, S11, S12, S26, S18 family, streptomyces trans- and carboxypeptiidases, signal peptidase I, Omtpin and Clp, C10C11, C15, C25 cysteine proteases, Porphyromonas gingivalis cyxteine proteases, sortase, metalloproteases of the thermolysin family (m4), Metalloproteases of the M9 family inclusive of vibrio and clostridium collagenases, Serralysin and related M10 Proteases and proteases of the M12 family, bacterial metallo exopeptidases, proteases of the M19, M20, M22, M23, and M26 families, tetanus and botulinum beurotoxins as being part of a group of bacterial metalloproteases, anthrax toxin lethal factor, lysostaphin and aureolysin, and AAA proteases.

In a preferred embodiment the pathogen is a pathogen listed in Table 1 of WO 2013/132058 A1, the content of which is incorporated herein in its entirety.

The protease-sensitive peptide may be selected from SEQ ID NOs:1-141 disclosed in WO 2013/132058 A1. The amino acid sequences of SEQ ID NOs:1-141 disclosed in WO 2013/132058 A1 are incorporated herein by reference.

In a particular embodiment, the protease-sensitive peptide comprises or consists of the amino acid sequence GPQGIAGQ.

In another embodiment the protease-sensitive peptide is not GPQGIAGA, DAPV or QPVV.

In yet another embodiment, the C-terminal amino acid of the protease-sensitive peptide is not alanine or valine.

In another embodiment the compound or salt of the invention does not comprise trifluoracetate (TFA). In another embodiment the salt of the invention comprises trifluoracetate (TFA).

In one embodiment, the compound of the invention is a compound of formula [BRS-X], wherein X stands for an amino acid:

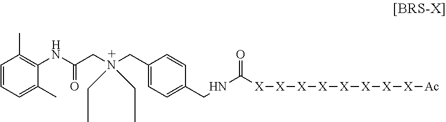

In a particular preferred embodiment, the compound of the invention is a compound of formula [BRS-1]:

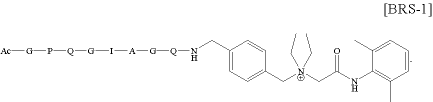

In another aspect the present invention relates to a salt of a compound of formula [I] as defined hereinabove. The salt typically comprises a nutritionally or pharmaceutically acceptable counter ion. The counter ion is preferably selected from the group consisting of
F⁻, Cl⁻, Br⁻,

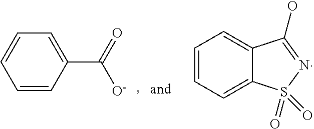 [SMILES: O=C([O-])c1ccccc1 ; O=S1(=O)N=C(O)c2ccccc21]

In yet another aspect the invention relates to a bioresponsive sensor comprising the compound of the present invention or the salt of the present invention. In a preferred embodiment the bioresponsive sensor does not comprise a base material or particles embedded and/or attached to said base material. In another preferred embodiment the bioresponsive sensor does not comprise particles. In another preferred embodiment the bioresponsive sensor substantially consists of the compound or salt of the invention.

In yet another aspect the invention relates to the compound, salt or bioresponsive sensor of the present invention for use in a method of detecting an inflammation in the oral cavity of a human patient. In accordance with the invention, the detection occurs directly in the oral cavity of the patient by the use of the human tongue (sense of taste) and the nose (sense of smell) as a detector, in particular by the sense of taste. If the patient suffers from an inflammation of the oral cavity, the bioresponsive diagnostic sensor comprised e.g. in a chewing gum comes into contact with the saliva containing the matrix metalloproteinases (MMP) long enough to react. Accordingly, it is possible for the patient himself to detect an inflammation in the oral cavity, e.g. periodontal disease and/or peri-implantitis by gustatory detection of a bitter taste. Therefore, a person using the inventive chewing gum knows when detecting a bitter taste, that an inflammation in the oral cavity, e.g. periodontal disease and/or peri-implantitis is present.

In another aspect the present invention provides a diagnostic chewing gum comprising the compound, salt or bioresponsive sensor of the present invention. The diagnostic chewing gum is typically used for detecting an inflammation, e.g. periodontal disease and/or peri-implantitis.

The present invention is further directed to a method of providing the inventive compound, salt or bioresponsive sensor. It is further directed to a method of providing a chewing gum comprising the inventive compound, salt or bioresponsive sensor.

According to the method of the invention for the preparation of a bioresponsive sensor, the protease-sensitive peptide is typically synthesized by solid-phase peptide synthesis (SPPS). Since sensory measurements with an electronic tongue showed that already four amino acids (AA) effectively mask the bitter taste of denatonium, no poly(methylmethacrylate) PMMA particles are needed anymore. The N-terminus of the protease-sensitive peptide is acetylated after the solid-phase peptide synthesis (SPPS) with acetic anhydride to prevent attack of the aminopeptidase (AP), and to prevent side reactions in further synthetic steps. Cleaved from the resin, the acetylated protease-sensitive peptide is now linked to Den-CH₂—NH₂ via 1-[Bis(dimethylamino)methylene]-1H-1,2,3-triazolo[4,5-b]pyridinium 3-oxide hexafluorophosphate (HATU). Thereby, a bioresponsive sensor with the general formula [BRS-x] is obtained, wherein X is selected from the group of the 20 proteinogenic amino acids independently from each other:

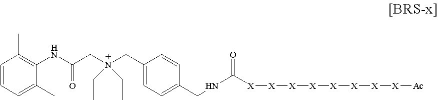

In another aspect, the present invention relates to a compound of formula [II]

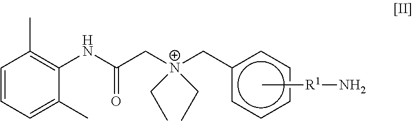

wherein

R¹ is an optionally substituted C₁₋₃ alkylene group. The preferred embodiments of R¹ in formula [II] correspond to the preferred embodiments of R¹ as defined above for formula [I].

The compound according to formula [II] can be coupled to a protease-sensitive peptide to provide the compound of formula [I]. When the compound according to formula [I] comes into contact with the saliva of a person having a disease of the oral cavity, e.g. periodontitis or peri-implantitis, the compound according to formula [II] is released.

Preferred embodiments of the compound according to formula [II] are selected from the following group consisting of [IIa] to [IIc], wherein [IIa] is particularly preferred:

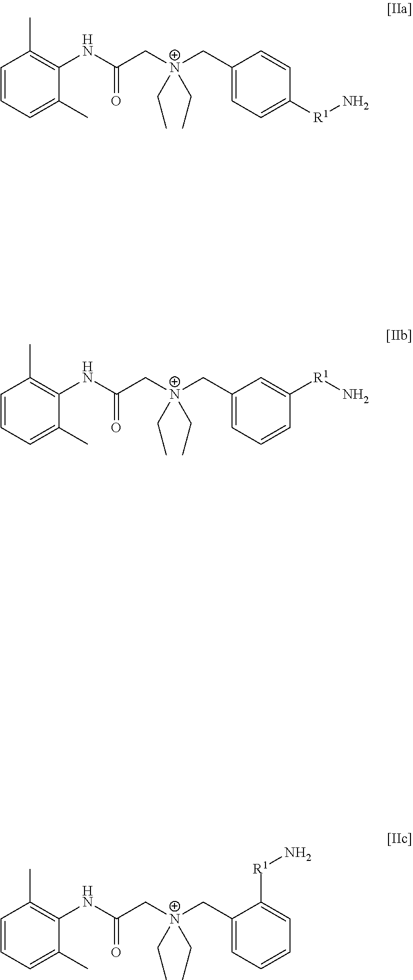

wherein

R¹ is an optionally substituted C₁₋₃ alkylene group.

In one embodiment, the compound of formula [IIa] is selected from the group consisting of [IIa-1] to [IIa-3], wherein formula [IIa-1] is particularly preferred:

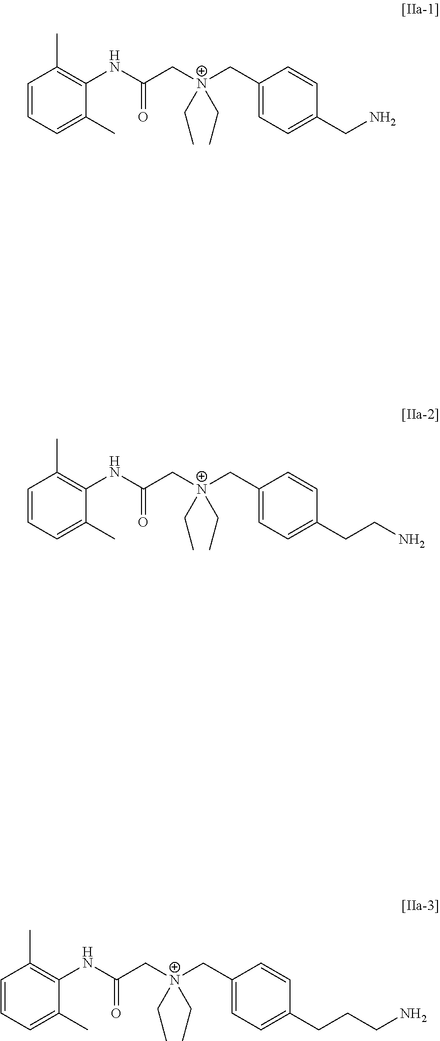 [SMILES: CC[N+](CC)(CC(=O)Nc1c(C)cccc1C)Cc1ccc(CN)cc1 ; CC[N+](CC)(CC(=O)Nc1c(C)cccc1C)Cc1ccc(CCN)cc1 ; CC[N+](CC)(CC(=O)Nc1c(C)cccc1C)Cc1ccc(CCCN)cc1]

In another embodiment, the bioresponsive sensor according to formula [IIb] is selected from the group consisting of [IIb-1] to [IIb-3]:

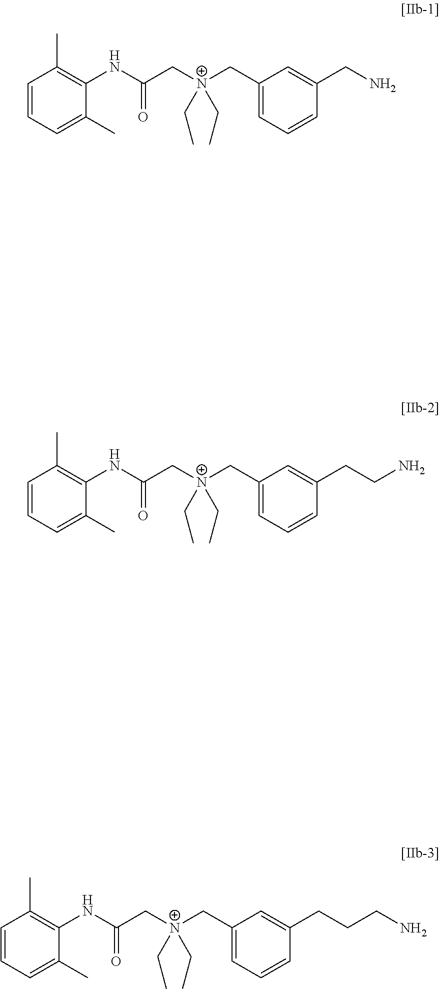 [SMILES: CC[N+](CC)(CC(=O)Nc1c(C)cccc1C)Cc1cccc(CN)c1 ; CC[N+](CC)(CC(=O)Nc1c(C)cccc1C)Cc1cccc(CCN)c1 ; CC[N+](CC)(CC(=O)Nc1c(C)cccc1C)Cc1cccc(CCCN)c1]

In another embodiment, the bioresponsive sensor according to formula [IIc] is selected from the group consisting of [IIc-1] to [IIc-3]:

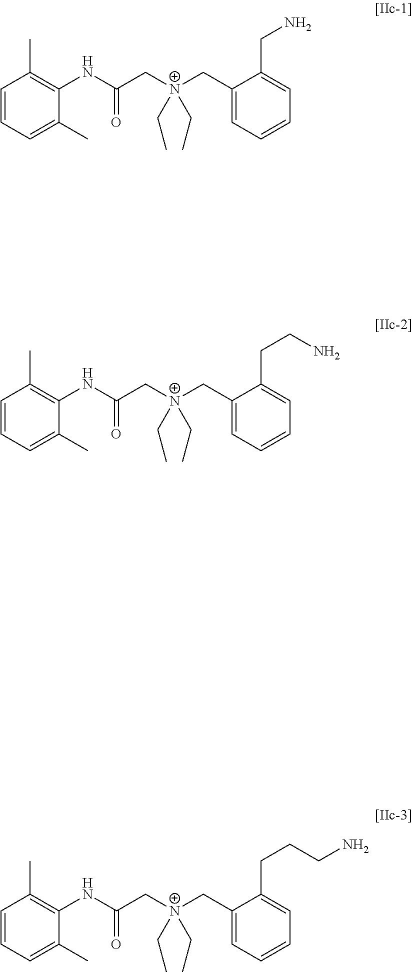 [SMILES: CC[N+](CC)(CC(=O)Nc1c(C)cccc1C)Cc1ccccc1CN ; CC[N+](CC)(CC(=O)Nc1c(C)cccc1C)Cc1ccccc1CCN ; CC[N+](CC)(CC(=O)Nc1c(C)cccc1C)Cc1ccccc1CCCN]

In a preferred embodiment the invention relates to a compound having the formula [IIa-1]:

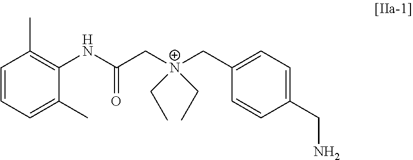 [SMILES: CC[N+](CC)(CC(=O)Nc1c(C)cccc1C)Cc1ccc(CN)cc1]

The compound is a stable Den-CH₂—NH₂ compound which comprises a methylamine group with which it can be attached to the peptide chain of a protease-sensitive peptide.

### EXAMPLES

### Example 1: Synthesis of Denatonium-CH₂—NHBoc

1.2 g lidocain (5.2 mmol) and 1.0 g tert-butyl-4-(bromomethyl)benzylcarbamate (3.4 mmol) were heated to 80° C. until the formation of a yellow melt. The melt then became highly viscous, up to solid, and subsequently, after a resting period of 10 minutes at 80° C., the obtained yellow solid was treated with 40 mL of a mixture of ethyl acetate and n-hexane (1:1). To obtain the protected product, the yellow solid was stirred for 10 minutes at 80° C. in the mixture. The resulting white residue was filtered off and washed with a mixture of ethyl acetate and hexane (1:1). 1.6 g of the protected product were obtained.

Sum formula: C₂₇H₄₀N₃O₃⁺Br⁻

Molecular mass: 534.5 g/mol

Yield: 1.6 g (91%).

### Example 2: Synthesis of a Denatonium-CH₂—NH₂ Salt

For the subsequent deprotection, 228 mg of the product (0.4 mmol) obtained from Example 1 was dissolved in 1 mL of trifluoroacetic acid and shaken at room temperature for 1 hour. The raw product was precipitated in diethylether and filtered off. The product was purified by chromatography.

Sum formula: C₂₂H₃₃N₃O²⁺2 C₂F₃O₂⁻

Molecular mass: 581.6 g/mol

Yield: 175 mg (75%).

¹H-NMR (DMSO, δ [ppm], J [Hz]): δ 10.14 (s, 1H), 8.30 (s, 3H), 7.62 (m, 4H), 7.20-7.10 (m, 3H), 4.84 (s. 2H), 4.18 (s, 2H), 4.12 (q. ³J_{H,H}=5.8, 2H), 3.54-3.48 (m, 4H), 2.20 (s, 6H), 1.42 (t, ³J_{H,H}=7.1, 6H).

¹³C-NMR (DMSO, δ [ppm], J [Hz]): δ 162.7 (s, 1C), 136.8 (s, 1C), 135.5 (s, 2C), 133.7 (s, 1C), 133.6 (s. 2C), 129.9 (s, 2C), 128.5 (s, 2C), 128.2 (s, 1C), 127.7 (s, 1C), 61.6 (s, 1C), 56.0 (s, 1C), 54.9 (s, 2C), 42.2 (s, 1C), 18.6 (s, 2C), 8.3 (s, 2C).

### Example 3

The protease-sensitive peptide was synthesized using solid-phase peptide synthesis to 2-chlorotrityl chloride resin. For the proof-of-concept of the system, the amino acid sequence GPQGIAGQ was prepared.
H-G-P-Q-G-I-A-G-Q-OH
   [PSP-1] (m/z=726.37)

This sequence was acetylated at the N-terminus according to a method known per se, so that a protected protease-sensitive peptide 2 [PSP-2] was obtained:
Ac-G-P-Q-G-I-A-G-Q-OH
   [PSP-2] (m/z=768.38)

Using HATU/DIPEA, [PSP-2] was then linked to compound [IIa-1]. Thereby the bioresponsive sensor 1 [BRS-1] was obtained.

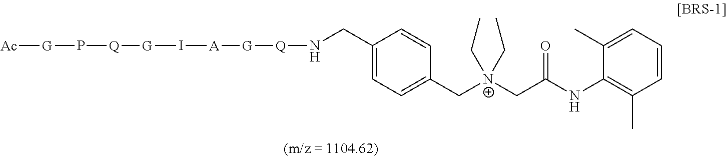

### Example 4: Cleavage Experiments

After incubation of the construct with MMP 1, 8 and 9, the following cleavage products CP1 and CP2 were obtained.

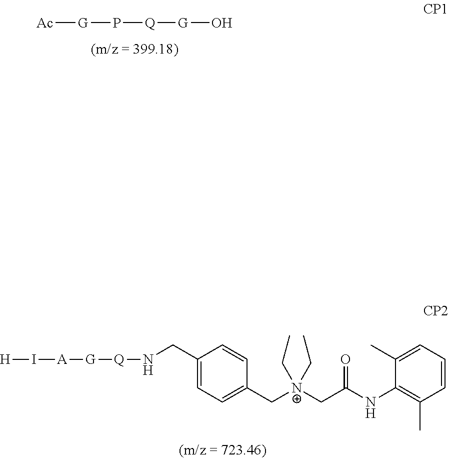

In the cleavage experiments, compound [BRS-1] was incubated with an equimolar mixture of MMP 1, 8 and 9 at 37° C. in MMP buffer (200 mM NaCl, 50 mM Tris-HCl, 5 mM CaCl₂), 1 mM ZnCl₂, 0.05% Brij 35, pH 7.0).

For comparison, a comparative construct [CC1], according to the prior art (Ritzer, J et al. Nat. Commun. volume 8, Article number: 264 (2017)) was incubated to evaluate the cleaving efficiency of the new system. In [CC1] denatonium is connected via a carboxylic acid group to the N-terminus of a protease-sensitive peptide, as given below:

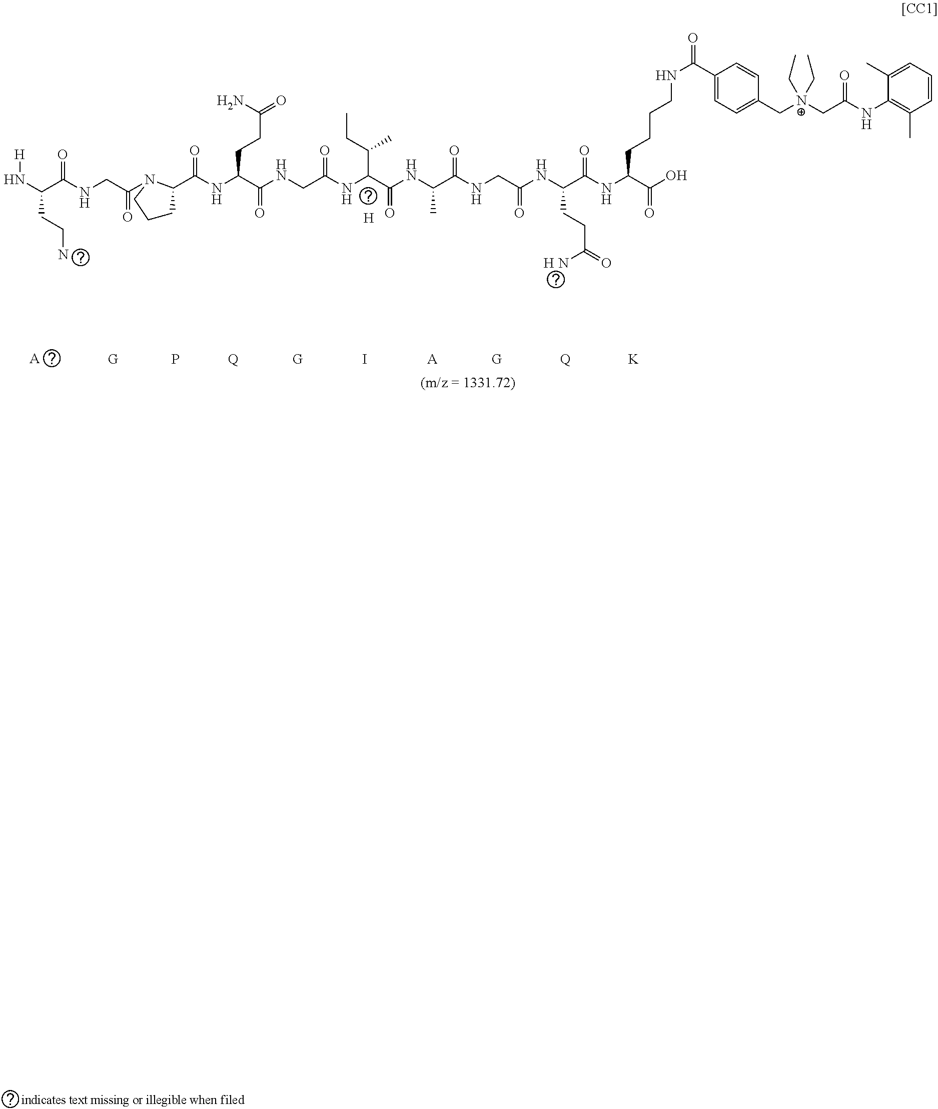

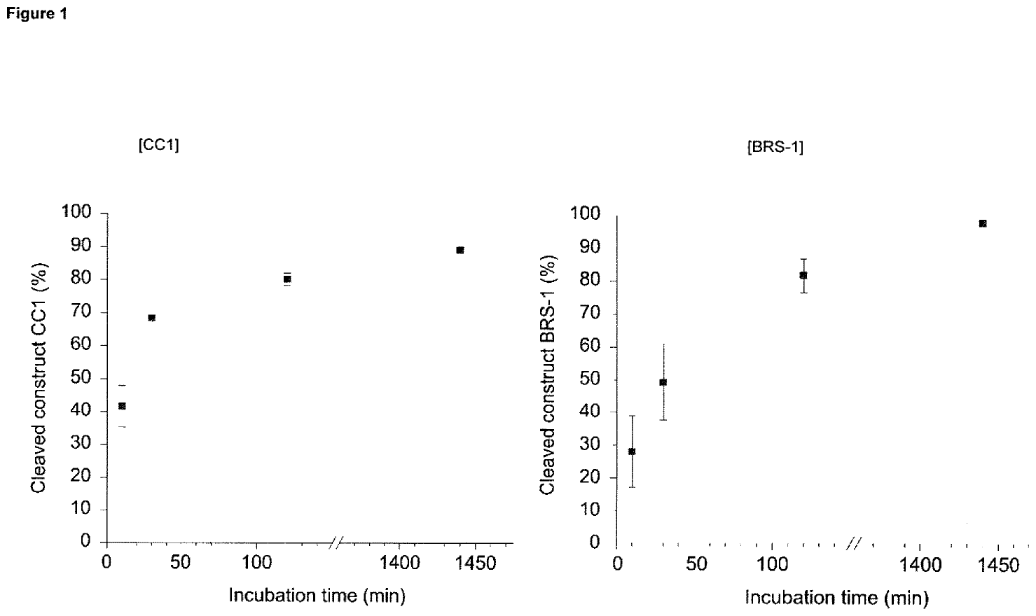

The concentration of the two compounds was 0.1 mM. The cleavage efficiency was measured using a Hitachi Elite LaChrom HPLC system (VWR, Darmstadt, Germany) with a ZORBAX Eclipse XDB-C18 column (4.6 mm internal diameter, 150 mm length (Agilent, Santa Clara, Calif.)), eluent A (0.1%). TFA in water, (v/v)) and eluent B (0.1% trifluoroacetic acid (TFA) in acetonitrile (v/v)) with a gradient of 5 to 95% eluent B over 55 min. The UV absorption was measured at I=214 nm (Table 1, FIG. 1).

TABLE 1
cleavage rate (%) of [BRS-1] and [CC1]
accompanied by the standard deviations.

| | [BRS-1] | [CC1] |
|---|---|---|
| Cleaved construct after 10 min | 28.0% ± 10.9% | 41.6% ± 6.29% |
| Cleaved construct after 30 min | 49.3% ± 11.7% | 68.4% ± 0.587% |
| Cleaved construct after 120 min | 81.8% ± 5.09% | 80.2% ± 1.78% |
| Cleaved construct after 120 min | 98.0% ± 0.0583% | 89.2% ± 0.888% |

The cleavage efficiency of both systems was comparable. The slightly better cleavage rate of [CC1] is caused by the fact that it was not the final construct that was analyzed here. Due to the lack of coupling with a spatially demanding PMMA particle, the overall system is further enlarged and thus less susceptible to MMP. On the other side, [BRS-1] is the final construct used as bioresponsive sensor in the detection of inflammations in the oral cavity.

When [CP2] is obtained after cleavage of [BRS-1] it is now possible for the aminopeptidase to attack the free N-terminus of [CP2], so that further cleavage to [IIa-1] occurs. Previously, in the full construct [BRS-1] this further cleavage process is prevented by the acetyl protecting group. Therefore, no degradation occurs in [BRS-1], when no MMP is present as it is the case in healthy individuals. [CC1] is cleaved by aminopeptidase analogously. However, in the case of [CC1] an amino acid (in this case lysine) remains on the denatonium-COOH, since it is attached to the ε-position. This significantly reduces the gustatory perception compared to free denatonium.

To trigger maximum gustatory perception, the last amino acid from the modified denatonium must also be removed by the aminopeptidase. Not all amino acids are equally well suited for this purpose. To evaluate the cleavage efficiency, 20 constructs consisting of one proteinogenic amino acid coupled to compound [IIa-1] were synthesized, thereby obtaining compound [Ia-X], wherein X is an amino acid:

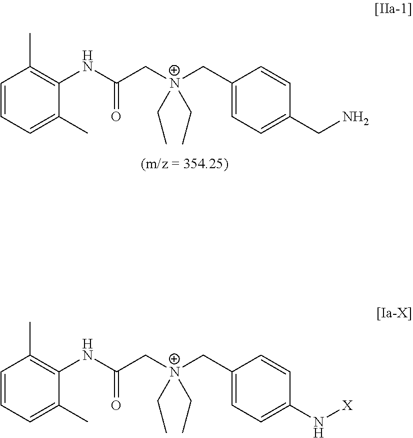

The resulting constructs were incubated with AP for 24 h and analyzed by LC/MS for complete degradation (Table 2).

TABLE 2
Cleavage Experiments of different compounds with AP
und their detectability after 24 h. + stands for
detectability, − stands for full degradation to [IIa-1].

| | Detectable after 24 h |
|---|---|
| [Ia-X] with X= | incubation with AP |
| Alanine | − |
| Arginine | − |
| Asparagine | + |
| Aspartic acid | + |
| Cysteine | − |
| Glutamine | + |
| Glutamic acid | − |
| Glycine | + |
| Histidine | + |
| Isoleucine | + |
| Leucine | − |
| Lysine | − |
| Methionine | − |
| Phenylalanine | − |
| Proline | + |
| Serine | − |
| Threonine | + |
| Tryptophan | + |
| Tyrosine | − |
| Valine | + |

Subsequently, cleavage experiments were performed for 20 min, 120 min and 24 h with an aminopeptidase concentration of 940 ng/ml of compound Ia-X. Only 16 out of 20 constructs were used, as lysine, asparagine, cysteine, and histidine did not produce significant synthesis yields, which also would interfere with subsequent peptide coupling.

### Example 5: Synthesis Example

The invention is further exemplified by the following example of the synthesis of [BRS-1]:

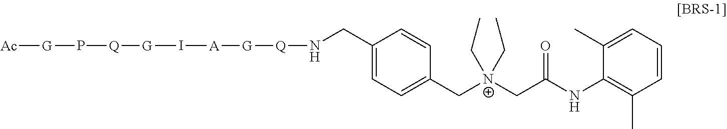

[PSP-2] (71.3 mg, 0.0928 mmol) was dissolved with HATU (34.0 mg, 0.0894 mmol) in anhydrous dimethylformamide (DMF) (2 mL). Subsequently, [IIa-1] (63.0 mg, 0.178 mmol) was added and after complete dissolution DIPEA (31.9 μL) was added. The mixture was stirred for 18 h protected from light at room temperature. Then ice-cold diethyl ether (20 mL) was added. After centrifugation and decantation of the supernatant, the residue was dried overnight.

The residue was purified using a FPLC system (Äkta purifier, GE Healthcare) using reversed phase chromatography (RPC) on a C18 column (Phenomenex®) (eluent A: 0.1% TFA in water, eluent B: 0.1% TFA in acetonitrile). After subsequent lyophilization, [BRS-1] was obtained as a colorless (white) powder. The overall yield of high purity [BRS-1] was 5.32 mg (5.19%).

1. A compound having the structure Den-R¹—NH—R², or a salt thereof,
wherein
Den is denatonium,
R¹ is an optionally substituted C₁₋₃ alkylene group
R² is a protease-sensitive peptide characterized by a C-terminal amino acid that forms an amide bond with the —NH— group of said compound.

2. The compound or salt of claim 1, having the formula [I]: 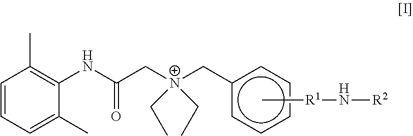
wherein
R¹ and R² are as defined in claim 1.

3. The compound or salt of claim 1, wherein the compound has the formula [Ia]: 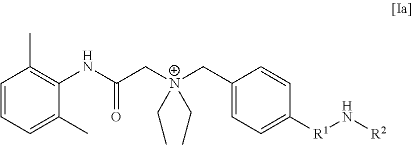
wherein
R¹ and R² are as defined in claim 1.

4. The compound or salt of claim 1, wherein R¹ is —CH₂—.

5. The compound or salt of claim 1, wherein the protease-sensitive peptide comprises at least 4 amino acids.

6. The compound or salt of claim 1, wherein the C-terminal amino acid of the protease-sensitive peptide is selected from the group consisting of alanine, arginine, glutamine, leucine, methionine and phenylalanine.

7. The compound or salt of claim 1, wherein the compound has the formula [BRS-1]:

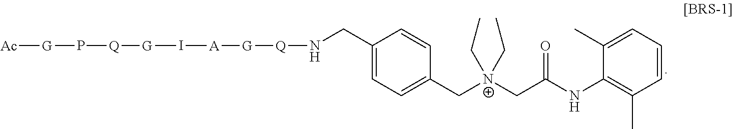

8. The compound or salt of claim 1, wherein the protease is a pathogen-specific protease.

9. The compound or salt of claim 1, wherein the protease-sensitive peptide is not QPVV, DAPV or GPQGIAGA.

10. The compound or salt of claim 1, wherein said compound is a salt comprising a counter ion selected from the group consisting of F⁻, Cl⁻, Br⁻,

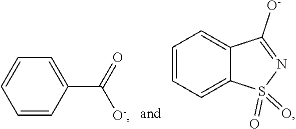

11. A bioresponsive sensor comprising the compound or salt of claim 1.

12. A method of detecting an inflammation in the oral cavity of a human patient comprising the step of applying a compound or salt according to claim 1 or a bioresponsive sensor comprising such a compound or salt to the oral cavity of said human patient.

13. A method for the preparation of the bioresponsive sensor according to claim 11, said method comprising the step of linking an amino-modified denatonium compound to the C-terminus of a protease-sensitive peptide.

14. The method of claim 13, wherein said method further comprises the performance of one or both of the following steps prior to said linking step:
preparing a protease-sensitive peptide by solid phase peptide synthesis, and
protecting the N-terminus of the protease-sensitive peptide with a protecting group, preferably acetic anhydride.

15. A diagnostic chewing gum or a diagnostic confectionary comprising the compound or salt of claim 1.

16. The compound or salt of claim 5, wherein the prof ease-sensitive peptide comprises 4 to 15 amino acids.

17. The compound or salt of claim 5, wherein the protease-sensitive peptide comprises 5 to 12 amino acids.

18. The compound or salt of claim 5, wherein the protease-sensitive peptide comprises 6 to 10 amino acids.

19. The compound or salt of claim 5, wherein the protease-sensitive peptide consists of 7 to 9 amino acids.

20. The method according to claim 13, wherein the amino-modified denatonium compound is a compound of formula [II] or formula [IIa-1].


SEQUENCE LISTING (11 sequences)

SEQ ID NO: 1 - protein, 4 aa, Artificial Sequence
  Description of Artificial Sequence Synthetic peptide
  peptide
QPVV

SEQ ID NO: 2 - protein, 4 aa, Artificial Sequence
  Description of Artificial Sequence Synthetic peptide
  peptide-1
DAPV

SEQ ID NO: 3 - protein, 8 aa, Artificial Sequence
  Description of Artificial Sequence Synthetic peptide
  peptide-2
GPQGIAGA

SEQ ID NO: 4 - protein, 8 aa, Artificial Sequence
  Description of Artificial Sequence Synthetic peptide
  BRS-1
  N-term Acetylation
  C-term Denatonium (Den), linked via amide bond
GPQGIAGQ

SEQ ID NO: 5 - protein, 8 aa, Artificial Sequence
  Description of Artificial Sequence Synthetic peptide
  PSP
GPQGIAGQ

SEQ ID NO: 6 - protein, 8 aa, Artificial Sequence
  Description of Artificial Sequence Synthetic peptide
  BRS-X
  N-term Acetylation
  MOD_RES (1)..(8): Xaa is selected from the group of the 20 proteinogenic amino acids independently from each other
  C-term Denatonium (Den), linked via amide bond
XXXXXXXX

SEQ ID NO: 7 - protein, 8 aa, Artificial Sequence
  Description of Artificial Sequence Synthetic peptide
  PSP-1
GPQGIAGQ

SEQ ID NO: 8 - protein, 8 aa, Artificial Sequence
  Description of Artificial Sequence Synthetic peptide
  PSP-2
  N-term acetylation
GPQGIAGQ

SEQ ID NO: 9 - protein, 4 aa, Artificial Sequence
  Description of Artificial Sequence Synthetic peptide
  CP1
  N-term acetylation
GPQG

SEQ ID NO: 10 - protein, 4 aa, Artificial Sequence
  Description of Artificial Sequence Synthetic peptide
  CP2
  C-term Denatonium (Den), linked via amide bond
IAGQ

SEQ ID NO: 11 - protein, 10 aa, Artificial Sequence
  Description of Artificial Sequence Synthetic peptide
  CC1
  MOD_RES (1)..(1): L-Azidohomoalanine (Aha)
  MOD_RES (10)..(10): Xaa is Lys with Denatonium carboxylate attached to its epsilon-amino group
AGPQGIAGQX